(19) United States
(12) Patent Application Publication
(10) Pub. No.: US 2003/0130287 A1
(43) Pub. Date: Jul. 10, 2003

(54) Piperidine and piperazine derivatives which function as 5-ht2a receptor antagonists

(76) Inventors: Karl-August Ackermann, Ober-ramstadt (DE); Henning Boettcher, Darmstadt (DE); Helmut Pruecher, Heppenheim (DE); Christoph V. Amsterdam, Darmstadt (DE); Christoph Seyfried, Seeheim-Jugenheim (DE); Hartmug Greiner, Weiterstadt (DE); Gerd Bartoszyk, Wetierstadt (DE); Juergen Harting, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/169,399

(22) PCT Filed: Jan. 5, 2001
(86) PCT No.: PCT/EP01/00080

(30) Foreign Application Priority Data
Jan. 11, 2000 (DE) 100 00 739.2

(51) Int. Cl. A61K 31/496; A61K 31/4545; C 07D 4 1/02; C 07D 4 3/02

(52) U.S. Cl. 514/253.07; 514/254.09; 514/318; 514/317; 544/363; 544/373; 546/193; 546/216

(57) ABSTRACT

Compounds of the formula I

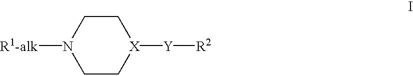

in which R¹, R², X, Y and alk are as defined in claim 1, are potent 5-HT_{2A} antagonists and are suitable for the treatment of psychoses, schizophrenia, depression, neurological disorders, memory disorders, Parkinson's disease, amyotrophic lateral sclerosis, Alzheimer's disease, Huntington's disease, eating disorders, such as bulimia, anorexia nervosa, premenstrual syndrome and/or for positively influencing obsessive-compulsive disorder (OCD).

The invention relates to compounds of the formula I

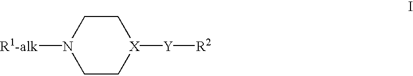

in which

R¹ and R² are each, independently of one another, a phenyl or naphthyl radical which is unsubstituted or substituted by R³, R⁴ and/or R⁵ or are Het¹,

R³, R⁴ and R⁵ are each, independently of one another, Hal, A, OA, OH, CN, NO₂, NH₂, NHA, NA₂, NH-acyl, acyl, —SA, —SOA, SO₂A, COOA or phenyl,

X is CH or N,

Y is SO₂ if X=N or

S, SO or SO₂ if X=CH,

Het¹ is an unsaturated heterocyclic ring system which is unsubstituted or monosubstituted, disubstituted or trisubstituted by Hal, A, CN, CONH₂, CH₂COOA, phenyl-SO₂, acyl, OA or OH and which contains one, two or three identical or different hetero atoms, such as nitrogen, oxygen and sulfur,

A is alkyl having 1-6 carbon atoms,

alk is alkylene having 1-6 carbon atoms, and

Hal is F, Cl, Br or I,

where Het¹≠2,1,3-benzoxadiazolyl or 2,1,3-benzothiadiazolyl,

and their physiologically acceptable salts and solvates.

The invention had the object of finding novel compounds having valuable properties, in particular those which can be used for the preparation of medicaments.

It has been found that the compounds of the formula I and their physiologically acceptable salts and solvates are well tolerated and have valuable pharmacological properties since they have actions on the central nervous system. The compounds have strong affinity to 5-HT_{2A} receptors, and they furthermore exhibit 5-HT_{2A} receptor-antagonistic properties.

For the in-vitro detection of affinity to 5-HT_{2A} receptors, the following test (Example A1), for example, can be used. The 5-HT_{2A} receptors are exposed both to [³H]ketanserine (a substance which is known for its affinity to the receptor) and also to the test compound. The decrease in the affinity of [³H]ketanserine to the receptor is an indication of the affinity of the test substance to the 5-HT_{2A} receptor. The detection is carried out analogously to the description by J. E. Leysen et al., Molecular Pharmacology, 1982, 21: 301-314, or as also described, for example, in EP 0320983.

The effectiveness of the compounds according to the invention as 5-HT_{2A} receptor antagonists can be measured in vitro analogously to W. Feniuk et al., Mechanisms of 5-hydroxytryptamine-induced vasoconstriction, in: The Peripheral Actions of 5-Hydroxytryptamine, ed. Fozard J R, Oxford University Press, New York, 1989, p. 110. Thus, the contractility of the rat tail artery caused by 5-hydroxytryptamine is mediated by 5-HT_{2A} receptors. For the test system, vessel rings prepared from the ventral rat tail artery are subjected to perfusion in an organ bath containing an oxygen-saturated solution. By introducing increasing concentrations of 5-hydroxytryptamine into the solution, a response is obtained to the cumulative concentration of 5-HT. The test compound is then added to the organ bath in suitable concentrations, and a second concentration curve for 5-HT is measured. The strength of the test compound in shifting the 5-HT-induced concentration curve to higher 5-HT concentrations is a measure of the 5-HT_{2A} receptor antagonistic property in vitro.

The 5-HT_{2A}-antagonistic property can be determined in vivo analogously to M. D. Serdar et al., Psychopharmacology, 1996, 128: 198-205.

Other compounds which likewise exhibit 5-HT₂-antagonistic actions are described, for example, in EP 0320983.

Differently substituted piperazine derivatives having antiarrhythmic properties are disclosed, for example, in EP 0431944 and EP 0431945.

Other indolecarbonyl derivatives having analgesic properties are described in EP 0599240. EP 0624584 describes piperazine derivatives as calmodoline inhibitors. WO 99/11641 describes phenylindole derivatives having 5-HT₂-antagonistic properties.

Differently substituted 4-(phenylsulfonyl)piperidine derivatives as active compounds against arrhythmia are described in EP 304888.

A. Morikawa et al. in Chem. Pharm. Bull. (1992), 40, 770-3, describe 5-isoquinolinesulfonamides as vasodilators.

H. Hidaka et al. in EP 61673 disclose other 5-isoquinolinesulfonamides as vasodilators.

M. Ohashi et al. in JP 63176177 describe piperazinesulfonyl derivatives as decolourizing agents.

The compounds of the formula I are suitable both in veterinary and in human medicine for the treatment of disturbances in the function of the central nervous system and of inflammations. They can be used for the prophylaxis and combating of the consequences of cerebral infarction phenomena (apoplexia cerebri), such as strokes and cerebral ischemia, and for the treatment of extrapyramidal motor side effects of neuroleptics and of Parkinson's disease, for the acute and symptomatic therapy of Alzheimer's disease and for the treatment of amyotrophic lateral sclerosis. They are likewise suitable as therapeutic agents for the treatment of brain and spinal traumas. In particular, however, they are suitable as medicament active ingredients for anxiolytics, antidepressants, antipsychotics, neuroleptics, antihypertonics and/or for positively influencing obsessive-compulsive disorder (OCD), anxiety states, panic attacks, psychoses, schizophrenia, anorexia, delusional obsessions, agoraphobia, migraines, Alzheimer's disease, sleep disturbances, tardive dyskinesia, learning disorders, age-dependent memory disorders, eating disorders, such as bulimia, drugs misuse and/or disturbances of sexual function. They are furthermore suitable for the treatment of endocrine illnesses, such as hyperprolactinemia, furthermore in vasospasms, hypertension and gastrointestinal illnesses.

They are furthermore suitable for the treatment of cardiovascular illnesses and extrapyramidal symptoms, as described in WO 99/11641, on page 2, lines 24-30.

The compounds according to the invention are furthermore suitable for reducing the intraocular pressure and for the treatment of glaucoma. They are also suitable for the prophylaxis and treatment of poisoning phenomena on administration of ergovaline to animals.

The compounds are furthermore suitable for the treatment of disorders of the cardiovascular system (WO 99/11641, page 3, lines 14-15). The compounds according to the invention can also be employed together with other active ingredients in the treatment of schizophrenia. Suitable other active ingredients are the compounds mentioned in WO 99/11641 on page 13, lines 20-26.

They can furthermore be employed as intermediates in the preparation of further medicament active ingredients.

The invention relates to the piperidine and piperazine derivatives of the formula I and to their physiologically acceptable acid-addition salts. The invention also relates to the solvates, for example hydrates or alcoholates, of these compounds.

Accordingly, the invention relates to the compounds of the formula I and to a process for the preparation of compounds of the formula I according to claim 1.

The process for the preparation of compounds of the formula I according to claim 1 in which X is N is characterized in that

a) a compound of the formula II

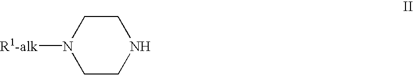

in which R¹ and alk are as defined in claim 1, is reacted with a compound of the formula III

R²—Y—L III

in which L is Cl, Br, I or a free or reactively functionally modified OH group, and R² and Y are as defined in claim 1,

or

b) if desired one of the radicals R¹ and/or R² is converted into another radical R¹ and/or R² by, for example, cleaving an OA group to form an OH group and/or converting a CHO group into a CN group,

and/or

a resultant base of the formula I is converted into one of its salts by treatment with an acid.

The process for the preparation of compounds of the formula I according to claim 1 in which X is CH is characterized in that

a) a compound of the formula IV

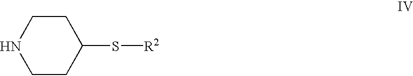

in which R² is as defined in claim 1, is reacted with a compound of the formula V

R¹-alk-L V

in which L is Cl, Br, I or a free or reactively functionally modified OH group, and R¹ and alk are as defined in claim 1,

and the product is subsequently oxidized,

or

b) if desired one of the radicals R¹ and/or R² is converted into another radical R¹ and/or R² by, for example, cleaving an OA group to form an OH group and/or converting a CHO group into a CN group,

and/or

a resultant base of the formula I is converted into one of its salts by treatment with an acid.

The invention also relates to the compounds of the formula I according to claim 1 and to their physiologically acceptable salts and solvates as medicaments.

The invention relates in particular to the compounds of the formula I

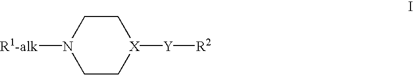

in which

R¹ and R² are each, independently of one another, a phenyl or naphthyl radical which is unsubstituted or substituted by R³, R⁴ and/or R⁵ or are Het¹,

R³, R⁴ and R⁵ are each, independently of one another, Hal, A, OA, OH, CN, NO₂, NH₂, NHA, NA₂, NH-acyl, acyl, —SA, —SOA, SO₂A, COOA or phenyl,

X is CH or N,

Y is SO₂ if X=N or

S, SO or SO₂ if X=CH,

Het¹ is an unsaturated heterocyclic ring system which is unsubstituted or monosubstituted, disubstituted or trisubstituted by Hal, A, CN, CONH₂, CH₂COOA, phenyl-SO₂, acyl, OA or OH and which contains one, two or three identical or different hetero atoms, such as nitrogen, oxygen and sulfur,

A is alkyl having 1-6 carbon atoms, alk is alkylene having 1-6 carbon atoms, and Hal is F, Cl, Br or I,

and their physiologically acceptable salts and solvates,

as medicaments having a 5-HT₂A receptor-antagonistic action.

The invention also relates to the compounds of the formula I and their enantiomers and diastereomers and to their salts.

For all radicals which occur more than once, such as, for example, A or Hal, their meanings are independent of one another.

The radical A is alkyl and has 1 to 6, preferably 1,2,3 or 4, in particular 1 or 2, carbon atoms. Alkyl is therefore in particular, for example, methyl, furthermore ethyl, n-propyl, isopropyl, n-butyl, sec-butyl or tert-butyl, furthermore also pentyl, 1-, 2- or 3-methylbutyl, 1,1-, 1,2- or 2,2-dimethyl-propyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl. In the said alkyl radicals, 1-7 H atoms may also be replaced by fluorine and/or chlorine. Thus, for example, A is also trifluoromethyl or pentafluoraethyl. Acyl preferably has 1-6 carbon atoms and is, for example, formyl, acetyl, propionyl, butyryl, furthermore trifluoroacetyl.

Alkylene has 1, 2, 3, 4, 5 or 6 carbon atoms, is unbranched or branched and is preferably methylene, ethylene, propylene, butylene or pentylene. Alkylene is very particularly preferably ethylene.

OA is preferably methoxy, trifluoromethoxy, furthermore also ethoxy, n-propoxy, isopropoxy, n-butoxy, isobutoxy, sec-butoxy or tert-butoxy.

Hal is fluorine, chlorine, bromine or iodine, in particular fluorine or chlorine.

R¹ and R² are each, independently of one another, phenyl or naphthyl, each of which is unsubstituted or preferably—as stated—substituted by R³ and/or R⁴, in detail preferably phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-hydroxy-phenyl, o-, m- or p-nitrophenyl, o-, m- or p-(trifluoromethoxy)phenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p-chlorophenyl, o-, m- or p-(difluoromethoxy)phenyl, o-, m- or p-(fluoromethoxy)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3, dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3-bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2,4- or 2,5-dinitrophenyl, 2,4- or 3,4-dimethoxyphenyl, 3-nitro-4-chlorophenyl, 2,3,4-, 2,3,5-, 2,3,6-, 2,4,6- or 3,4,5-trichlorophenyl, 2,4,6-tri-tert-butylphenyl, furthermore preferably 2-nitro-4-(trifluoromethyl)phenyl, 3,5-di(trifluoromethyl)phenyl, 2,4-dimethylphenyl, 2-hydroxy-3,5-dichlorophenyl, 2-fluoro-5- or 4-fluoro-3-(trifluoromethyl)phenyl, 4-chloro-2- or 4-chloro-3-(trifluoromethyl)-, 2-chloro-4- or 2-chloro-5-(trifluoromethyl)phenyl, 4-bromo-2- or 4-bromo-3-(trifluoromethyl)phenyl, p-iodophenyl, 2-nitro-4-methoxyphenyl, 2,5-dimethoxy-4-nitrophenyl, 2-methyl-5-nitrophenyl, 2,4-dimethyl-3-nitrophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl.

R¹ and R² are also each, independently of one another, Het¹. Het¹ is preferably 2- or 3-furyl, 2- or 3-thienyl, 1-,2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4H-thiopyranyl, 3- or 5-pyridazinyl, pyrazinyl, 2,-, 3-, 4-, 5- 6- or 7-benzofuryl, 2-, 3-, 4-, 5-, 6- or 7-benzothienyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolyl, 2-, 4-, 5-, 6-, 7- or 8-quinazolinyl, 1-, 2-, 3- or 4-dibenzofuranyl, furthermore 3-methyl-3H-imidazo[4,5-c]pyridin-4-yl, 1-methyl-1H-imidazo[4,5-c]pyridin-4-yl, 1(3)-H-imidazo[4,5-c]pyridin-4-yl, imidazo[2,1-thiazol-5-yl or 2,3-dihydro-1H-indolyl.

R¹ is very particularly preferably phenyl, p-chlorophenyl, p-fluorophenyl, thiophen-2-yl, 5-chlorothiophen-2-yl, 2,5-dichlorothiophen-3-yl or 2- or 3-furyl.

R² is very particularly preferably 4-propylphenyl, 2-isopropylphenyl, butyl, 2,4,6-trimethylphenyl, 2- or 4-methoxyphenyl, 2-, 3- or 4-methoxycarbonyl-phenyl, 2-, 3- or 4-ethoxycarbonylphenyl, 2- or 4-chlorophenyl, 2-nitro-phenyl, 4′-biphenyl, 2,4,6-trimethylphenyl, 3,4-dimethylphenyl, 2-naphthyl, 6-chloro-2-naphthyl, 5-chloro-1-naphthyl, 5-dibutylamino-1-naphthyl, 4-isopropylphenyl, 2-thienyl, 2,1,3-benzothiadiazol-4-yl, 4-fluorophenyl, 2-chloropyridin-6-yl, 3,4-dimethoxyphenyl, 2,4-dichlorophenyl, 4-tolyl, 2,4-, 2,5- or 3,4-difluorophenyl, 2-fluorophenyl, 2-methoxypyridin-6-yl, quinolin-8-yl, isoquinolin-1-yl, 5-acetamidonaphth-1-yl, 5-dimethylaminonaphth-1-yl, dibenzofuran-1-yl, thiophen-2-yl, 4-phenylsulfonylthiophen-2-yl, 4-phenyl-sulfonylthiophen-3-yl, 5-chloro-3-methylbenzo[b]thiophen-2-yl, pyrimidin-2-yl, indol-3- or -5-yl, 3-cyanoindol-5-yl, benzimidazol-2-yl, 1-methyl-1H-imidazol-2-yl, 1-methyl-1H-tetrazol-5-yl, 4-methyl-4H-[1,2,4]triazol-3-yl, 4,5-dihydrothiazol-2-yl, 2-methoxycarbonylmethylthiazol-4-yl, benzo[2,1,3]-oxadiazol-4-yl, 1-acetyl-2,3-dihydroindol-5-yl, 2,3-dihydro-1H-indol-5-yl, 1-methyl-1H-imidazol-4-yl or 1-(3-chloro-5-trifluoromethylpyridin-2-yl)pyrrol-3-yl.

The invention also relates to the compounds 4-{4-[2-(4-fluorophenyl)ethyl]-piperazin-1-sulfonyl}-2,1,3-benzothiadiazole and 4-{4-[2-(4-fluorophenyl)-ethyl]piperazin-1-sulfonyl}-2,1,3-benzoxadiazole.

Accordingly, the invention relates in particular to the compounds of the formula I in which at least one of the said radicals has one of the preferred meanings indicated above. Some preferred groups of compounds may be expressed by the following subformulae Ia to Ik which correspond to the formula I and in which the radicals not denoted more precisely are as defined for the formula I, but in which

in Ia R¹ is a phenyl or naphthyl radical which is substituted by R³, R⁴ and/or R⁵ or Het¹;

in Ib R¹ is a phenyl or naphthyl radical which is substituted by R³, R⁴ and/or R⁵;

in Ic R¹ is a phenyl or naphthyl radical which is substituted by R³, R⁴ and/or R⁵,

R² is a phenyl or naphthyl radical which is substituted by R³, R⁴ and/or R⁵ or Het¹;

in Id R¹ is a phenyl or naphthyl radical which is substituted by R³, R⁴ and/or R⁵,

R² is a phenyl or naphthyl radical which is substituted by R³, R⁴ and/or R⁵ or Het¹,

Het¹ is an unsaturated heterocyclic ring system which is unsubstituted or monosubstituted or disubstituted by Hal, CN, acyl, phenyl-SO₂ or A and which contains one or two identical or different hetero atoms, such as nitrogen, oxygen and sulfur;

in Ie R¹ is a phenyl or naphthyl radical which is substituted by R³, R⁴ and/or R⁵,

R² is a phenyl or naphthyl radical which is substituted by R³, R⁴ and/or R⁵ or Het¹,

Het¹ is thienyl, dibenzofuranyl, benzo[b]thiophenyl, indolyl, pyridinyl, benzo[2,1,3]oxadiazol-4-yl, 2,3-dihydro-1H-indol-5-yl, imidazolyl or 1-(pyridin-2-yl)pyrrolyl, each of which is unsubstituted or monosubstituted or disubstituted by Hal, CN, acyl, phenyl-SO₂ or A;

in If X is CH,

R¹ is a phenyl or naphthyl radical which is unsubstituted or substituted by R³, R⁴ and/or R⁵,

R² is a phenyl or naphthyl radical which is unsubstituted or substituted by R³, R⁴ and/or R⁵ or Het¹,

R³, R⁴ and R⁵ are each, independently of one another, Hal, CN, —SA, A, COOA or OA,

Het¹ is thienyl, quinolinyl, isoquinolinyl, dibenzofuranyl, benzo[b]thiophenyl, tetrazolyl, triazolyl or imidazolyl, pyridinyl, 4,5-dihydrothiazolyl, pyrimidinyl, benzimidazolyl or indolyl, each of which is unsubstituted or monosubstituted or disubstituted by Hal, A or CH₂COOA;

in Ig X is CH,

R¹ is a phenyl radical which is substituted by R³, R⁴ and/or R⁵,

R² is a phenyl radical which is substituted by R³, R⁴ and/or R⁵,

R³, R⁴ and R⁵ are each, independently of one another, Hal, CN, —SA, A, COOA or OA,

alk is alkylene having 1-4 carbon atoms;

in Ih X is N,

R¹ is a phenyl or naphthyl radical which is unsubstituted or substituted by R³, R⁴ and/or R⁵,

R² is a phenyl or naphthyl radical which is unsubstituted or substituted by R³, R⁴ and/or R⁵ or Het¹,

R³, R⁴ and R⁵ are each, independently of one another, Hal, A, OA, NH₂, NHA, NA₂, NH-acyl, acyl or phenyl,

Het¹ is an unsaturated heterocyclic ring system which is unsubstituted or monosubstituted or disubstituted by Hal, CN, acyl, phenyl-SO₂ or A and which contains one or two identical or different hetero atoms, such as nitrogen, oxygen and sulfur;

in Ii X is N,

R¹ is a phenyl or naphthyl radical which is unsubstituted or substituted by R³, R⁴ and/or R⁵,

R² is a phenyl or naphthyl radical which is unsubstituted or substituted by R³, R⁴ and/or R₅ or Het¹,

R³, R⁴ and R⁵ are each, independently of one another, Hal, A, OA, NH₂, NHA, NA₂, NH-acyl, acyl or phenyl,

Het¹ is thienyl, dibenzofuranyl, benzo[b]thiophenyl, indolyl, pyridinyl, benzo[2,1,3]oxadiazol-4-yl, 2,3-dihydro-1H-indol-5-yl, imidazolyl or 1-(pyridin-2-yl)pyrrolyl, each of which is unsubstituted or monosubstituted or disubstituted by Hal, CN, acyl, phenyl-SO₂ or A;

in Ij X is N,

R¹ is a phenyl or naphthyl radical which is unsubstituted or substituted by R³, R⁴ and/or R⁵,

R² is a phenyl or naphthyl radical which is unsubstituted or substituted by R³, R⁴ and/or R⁵ or Het¹,

R³, R⁴ and R⁵ are each, independently of one another, Hal, A, OA, NH₂, NHA, NA₂, NH-acyl, acyl or phenyl,

Het¹ is thienyl, dibenzofuranyl, benzo[b]thiophenyl, indolyl, pyridinyl, benzo[2,1,3]oxadiazol-4-yl, 2,3-dihydro-1H-indol-5-yl, imidazolyl or 1-(pyridin-2-yl)pyrrolyl, each of which is unsubstituted or monosubstituted or disubstituted by Hal, CN, acyl, phenyl-SO₂ or A;

in Ik X is CH or N,

R¹ is a phenyl radical which is unsubstituted or substituted by R³, R⁴ and/or R⁵,

R² is a phenyl radical which is unsubstituted or substituted by R³, R⁴ and/or R⁵

R³, R⁴ and R⁵ are each, independently of one another, Hal, A, COOA or OA,

alk is alkylene having 1-4 carbon atoms;

where Het¹≠2,1,3-benzoxadiazolyl or 2,1,3-benzothiadiazolyl,

and their physiologically acceptable salts and solvates.

The compounds of the formula I and also the starting materials for their preparation are, in addition, prepared by methods known per se, as described in the literature (for example in standard works such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for said reactions. Use can also be made here of variants which are known per se, but are not mentioned here in greater detail.

The starting materials for the claimed process can, if desired, also be formed in situ by not isolating them from the reaction mixture, but instead immediately converting them further into the compounds of the formula I. On the other hand, it is possible to carry out the reaction stepwise.

In the compounds of the formulae III and V, the radical L is preferably Cl or Br; however, it can also be I, OH or also preferably a reactively functionally modified OH group, in particular alkylsulfonyloxy having 1-6 carbon atoms (for example methanesulfonyloxy) or arylsulfonyloxy having 6-10 carbon atoms (for example benzenesulfoniloxy, p-toluenesulfonyloxy, 1- or 2-naphthylenesulfonyloxy) or alternatively trichloromethoxy, alkoxy, such as, for example, methoxy, ethoxy, propoxy or butoxy, furthermore also phenoxy.

The compounds of the formula I in which X is N can preferably be obtained by reacting compounds of the formula II with compounds of the formula III.

The starting materials of the formulae II and III are generally known; the unknown compounds of the formulae II and III can easily be prepared analogously to the known compounds.

The reaction of the compounds II and III proceeds by methods as known from the literature for the alkylation or acylation of amines. However, it is also possible to react the compounds in the presence of an inert solvent. Examples of suitable solvents are hydrocarbons, such as benzene, toluene or xylene; ketones, such as acetone or butanone; alcohols, such as methanol, ethanol, isopropanol or n-butanol; ethers, such as tetrahydro-furan (THF) or dioxane; amides, such as dimethylformamide (DMF) or N-methylpyrrolidone; nitrites, such as acetonitrile, optionally also mixtures of these solvents with one another or mixtures with water. The addition of an acid-binding agent, for example an alkali metal or alkaline earth metal hydroxide, carbonate or bicarbonate or of another salt of a weak acid of the alkali metals or alkaline earth metals, preferably of potassium, sodium or calcium, or the addition of an organic base, such as triethylamine, dimethylaniline, pyridine or quinoline, or of an excess of piperazine derivative of the formula II, may be favourable. Depending on the conditions used, the reaction time is between a few minutes and 14 days, and the reaction temperature is between about 0 and 150°, normally between 20 and 130°.

Furthermore, compounds of the formula I in which X is CH can be prepared by reacting amines of the formula IV with a component of the formula V, and subsequently oxidizing the reaction product. The oxidation generally gives a mixture of sulfinyl and sulfonyl compounds, which can be separated into the individual compounds by chromatography or by crystallization.

The respective components are generally known or can be prepared by known processes as already described. The reaction between the compounds of the formulae IV and V proceeds under conditions as described for the reaction between the compounds of the formulae II and III.

The resultant base of the formula I can be converted into the associated acid-addition salt using an acid. Suitable acids for this reaction are those which give physiologically acceptable salts. Thus, it is possible to use inorganic acids, for example sulfuric acid, hydrohalic acids, such as hydrochloric acid or hydrobromic acid, phosphoric acids, such as ortho-phosphoric acid, nitric acid, sulfamic acid, furthermore organic acids, in detail aliphatic, alicyclic, araliphatic, aromatic or heterocyclic monobasic or polybasic carboxylic, sulfonic or sulfuric acids, such as formic acid, acetic acid, propionic acid, pivalic acid, diethylacetic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methane- or ethanesulfonic acid, ethanedisulfonic acid, 2-hydroxyethanesulfonic acid; benzenesulfonic acid, p-toluenesulfonic acid, naphthalenemono- and -disulfonic acids and laurylsulfuric acid.

The free bases of the formula I may, if desired, be liberated from their salts by treatment with strong bases, such as sodium hydroxide, potassium hydroxide, sodium carbonate or potassium carbonate, so long as the molecule contains no further acidic groups. In those cases where the compounds of the formula I have free acid groups, salt formation can likewise be achieved by treatment with bases. Suitable bases are alkali metal hydroxides, alkaline earth metal hydroxides or organic bases in the form of primary, secondary or tertiary amines.

The invention furthermore relates to the medicaments according to the invention having a 5-HT_{2A} receptor-antagonistic action for the treatment of psychoses, schizophrenia, depression, neurological disorders, memory disorders, Parkinson's disease, amyotrophic lateral sclerosis, Alzheimer's disease, Huntington's disease, eating disorders, such as bulimia, anorexia nervosa, premenstrual syndrome and/or for positively influencing obsessive-compulsive disorder (OCD).

The invention also relates to a pharmaceutical preparation comprising at least one medicament according to the invention and optionally vehicles and/or auxiliaries and optionally other active ingredients. The medicaments here can be brought into a suitable dosage form together with at least one solid, liquid and/or semiliquid vehicle or auxiliary and optionally in combination with one or more further active ingredient(s).

The invention furthermore relates to the use of the compounds according to the invention and/or of their physiologically acceptable salts and solvates for the preparation of a medicament having a 5-HT_{2A} receptor-antagonistic action.

The invention also relates to the use of the compounds according to the invention and/or their physiologically acceptable salts and solvates for the preparation of a medicament having a 5-HT_{2A} receptor-antagonistic action for the treatment of psychoses, schizophrenia, depression, neurological disorders, memory disorders, Parkinson's disease, amyotrophic lateral sclerosis, Alzheimer's disease, Huntington's disease, eating disorders, such as bulimia, anorexia nervosa, premenstrual syndrome and/or for positively influencing obsessive-compulsive disorder (OCD).

The pharmaceutical preparations can be employed as medicaments in human and veterinary medicine. Suitable vehicles are organic or inorganic substances which are suitable for enteral (for example oral), parenteral or topical application and do not react with the novel compounds, for example water, vegetable oils, benzyl alcohols, polyethylene glycols, gelatin, carbohydrates, such as lactose or starch, magnesium stearate, talc or vaseline. Suitable for enteral administration are, in particular, tablets, coated tablets, capsules, syrups, juices, drops or suppositories, suitable for parenteral application are solutions, preferably oily or aqueous solutions, furthermore suspensions, emulsions or implants, and suitable for topical application are ointments, creams or powders. The novel compounds may also be lyophilized and the resultant lyophilizates used, for example, for the preparation of injection preparations.

The preparations indicated may be sterilized and/or comprise auxiliaries, such as lubricants, preservatives, stabilizers and/or wetting agents, emulsifiers, salts for modifying the osmotic pressure, buffer substances, dyes, flavours and/or aroma substances. If desired, they may also comprise one or more further active ingredients, for example one or more vitamins.

The substances according to the invention are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg of body weight.

The substances according to the invention are generally administered in doses of between about 1 and 500 mg, in particular between 5 and 100 mg, per dosage unit. The daily dose is preferably between about 0.02 and 10 mg/kg of body weight. However, the specific dose for each particular patient depends on a wide variety of factors, for example on the efficacy of the specific compound employed, on the age, body weight, general state of health, sex, on the diet, on the time and method of administration, on the rate of excretion, medicament combination and severity of the particular illness to which the therapy applies. Oral administration is preferred.

Above and below, all temperatures are given in ° C. In the examples below, “conventional work-up” means that the solvent is removed if necessary, water is added if necessary, the mixture is adjusted, if necessary, to a pH of between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallization.

### EXAMPLE A1

Preparation of a Suspension of 5-HT_{2A} Receptors:

Frontal rat cortex is homogenized in ice-cold buffer. The homogenate is centrifuged for 10 minutes at 4° C. and 50,000 X. The pellet is re-suspended in 2.5 ml of ice-cold tris buffer, made up to 10 ml with additional buffer and centrifuged as described. The pellet is then re-suspended in buffer and diluted to give a homogenate comprising 60 mg of material/ml. 0.1 ml of the suspension, 100 μl of a 5 nM solution of [³H]ketanserine and 100 μl of a solution of the test compound (concentration in the range from 10⁻⁵ to 10⁻¹⁰ mol per litre) are introduced into the incubation tubes and made up to 1 ml with buffer. The tubes are incubated for 15 minutes at 37° C. After the incubation has been terminated by dipping the tubes into an ice bath, the cooled suspension is filtered through a glass filter under reduced pressure. The filters are washed 3× with 5 ml of cold buffer and then transferred into scintillation tubes. The filters are analysed by liquid scintillation spectrometry in 8 ml of Triton-X scintillator liquid.

Test Results

1. 4-(8-Quinolinesulfonyl)-1-[2-(4-fluorophenyl)ethyl]piperazine, hydrochloride: IC50 (5-HT_{2A})=1.3 nM/I.

2. 4-(1-Naphthylsulfonyl)-1-[2-(4-fluorophenyl)ethyl]piperazine, hydrochloride: IC50 (5-HT_{2A})=8.1 nM/I.

3. 4-(4-Fluorophenylsulfonyl)-1-[2-(4-fluorophenyl)ethyl]piperazine, hydrochloride: IC50 (5-HT_{2A})=25.0 nM/I.

4. 4-(5-Acetamidonaphth-1-yl-sulfonyl)-1-[2-(4-fluorophenyl)ethyl]piperazine, hydrochloride: IC50 (5-HT_{2A})=7.4 nM/I.

5. 4-(2,1,3-Benzoxadiazol-4-ylsulfonyl)-1-[2-(4-fluorophenyl)ethyl]piperazine, hydrochloride: IC50 (5-HT_{2A})=44.0 nM/I.

6. 4-(2-Nitrophenylsulfonyl)-1-[2-(4-fluorophenyl)ethyl]piperazine, hydrochloride: IC50 (5-HT_{2A})=9.2 nM/I.

7. 4-(2,3-Dihydro-1H-indole-5-sulfonyl)-1-[2-(4-fluorophenyl)ethyl]piperazine, hydrochloride: IC50 (5-HT_{2A})=13.0 nM/I.

8. 4-(3-Cyano-1H-indole-5-sulfonyl)-1-[2-(4-fluorophenyl)ethyl]piperazine: IC50 (5-HT2)=1.6 nM/I.

9. 4-(4-Fluorophenylsulfonyl)-1-(2-(4-fluorophenyl)ethyl]piperidine, hydrochloride: IC50 (5-HT_{2A})=2.1 nM/I. 10. 2-Chloro-6-{1-[2-(4-fluorophenyl)ethyl]piperidin-4-sulfonyl}pyridine, hydrochloride: IC50 (5-HT_{2A})=0.6 nM/I.

### Synthesis Examples

### Example 1

840 g of NaHCO₃ are added in portions at 40° to a solution of 590 g of BOC-(tert-butoxycarbonyl)piperazine and 700 g of 2-(4-fluorophenyl)ethyl methanesulfonate, and the mixture is subsequently refluxed for 12 hours. After the mixture has been cooled and filtered, it is subjected to conventional work-up, giving 1013 g of 1-BOC-4-[2-(4-fluorophenyl)ethyl]piperazine, m.p. 68-70°.

The compound is dissolved in 1500 ml of dioxane, and 400 ml of ethanolic hydrochloric acid are added. The mixture is refluxed for 12 hours. After the mixture has been cooled, the precipitated crystals are separated off, washed with dioxane and dried, giving 440 g of 1-[2-(4-fluorophenyl)ethyl]piperazine, dihydrochloride, (“AB”), m.p. 272-274°.

2.0 g of “AB” and 1.78 g of 8-chlorosulfonylquinoline are dissolved in 100 ml of dichloromethane, 6.0 g of polymer-immobilized 4-dimethylamino pyridine (DMAP on polystyrene) are added, and the mixture is stirred at room temperature for 24 hours. Filtration and conventional work-up give 1.2 g of 4-(8-quinolinesulfonyl)-1-[2-(4-fluorophenyl)ethyl]piperazine, hydrochloride, m.p. 141°.

The following compounds are obtained analogously:

4-(4-propylphenylsulfonyl)-1-(2-phenylethyl)piperazine,

4-(butylsulfonyl)-1-(2-phenylethyl)piperazine,

4-(4-methoxyphenylsulfonyl)-1-(2-phenylethyl)piperazine,

4-(4-chlorophenylsulfonyl)-1-(2-phenylethyl)piperazine,

4-(4-methoxyphenylsulfonyl)-1-(2-phenylethyl)piperazine,

4-(biphenyl-4-sulfonyl)-1-(2-phenylethyl)piperazine,

4-(2,4,6-trimethylphenylsulfonyl)-1-(2-phenylethyl)piperazine,

4-(2-phenylethenylsulfonyl)-1-(2-phenylethyl)piperazine,

4-(3-chloro-4-methylphenylsulfonyl)-1-(2-phenylethyl)piperazine,

4-(2-naphthylsulfonyl)-1-(2-phenylethyl)piperazine,

4-(6-chloronaphth-2-ylsulfonyl)-1-(2-phenylethyl)piperazine,

4-(4-methoxyphenylsulfonyl)-1-[2-(3,5-dimethoxyphenyl)ethyl]piperazine,

4-(4-isopropylphenylsulfonyl)-1-[2-(3,5-dimethoxyphenyl)ethyl]piperazine,

4-(biphenyl-4-sulfonyl)-1-[2-(3,5-dimethoxyphenyl)ethyl]piperazine,

4-(2-naphthylsulfonyl)-1-[2-(3,5-dimethoxyphenyl)ethyl]piperazine,

4-(6-chloronaphth-2-ylsulfonyl)-1-[2-(3,5-dimethoxyphenyl)ethyl]piperazine,

4-(2-thienylsulfonyl)-1-[2-(4-fluorophenyl)ethyl]piperazine, hydrochloride, m.p. 226-228°;

4-(1-naphthylsulfonyl)-1-[2-(4-fluorophenyl)ethyl]piperazine, hydrochloride, m.p. 231°;

4-(2,1,3-benzothiadiazol-4-ylsulfonyl)-1-[2-(4-fluorophenyl)ethyl]piperazine, hydrochloride, m.p. 207°;

4-(4-fluorophenylsulfonyl)-1-[2-(4-fluorophenyl)ethyl]piperazine, hydrochloride, m.p. 237°;

4-(5-acetamidonaphth-1-ylsulfonyl)-1-[2-(4-fluorophenyl)ethyl]piperazine, hydrochloride, m.p. 243°;

4-(5-dimethylaminonaphth-1-ylsulfonyl)-1-[2-(4-fluorophenyl)ethyl]piperazine, hydrochloride;

4-(5-chloronaphth-1-ylsulfonyl)-1-[2-(4-fluorophenyl)ethyl]piperazine, hydrochloride, m.p. 241°;

4-(dibenzofuran-1-ylsulfonyl)-1-[2-(4-fluorophenyl)ethyl]piperazine, hydrochloride, m.p. 216-217°;

4-(5-chloro-3-methylbenzo[b]thiophen-2-ylsulfonyl)-1-[2-(4-fluorophenyl) ethyl]piperazine, hydrochloride, m.p. 250°;

4-(5-dibutylaminonaphth-1-ylsulfonyl)-1-[2-(4-fluorophenyl)ethyl]piperazine, hydrochloride, m.p. 191°;

4-(2,1,3-benzoxadiazol-4-ylsulfonyl)-1-[2-(4-fluorophenyl)ethyl]piperazine, hydrochloride, m.p. 211-212°;

4-(2,5-difluorophenylsulfonyl)-1-[2-(4-fluorophenyl)ethyl]piperazine, hydrochloride, m.p. 244-247°;

4-(2-nitrophenylsulfonyl)-1-[2-(4-fluorophenyl)ethyl]piperazine, hydrochloride, m.p. 213-214°;

4-(2-aminophenylsulfonyl)-1-[2-(4-fluorophenyl)ethyl]piperazine, dihydrochloride/hydrate, m.p. 211-215°;

4-(3-cyano-1H-indole-5-sulfonyl)-1-[2-(4-fluorophenyl)ethyl]piperazine;

4-(4-phenylsulfonylthiophene-2-sulfonyl)-1-[2-(4-fluorophenyl)ethyl]piperazine, hydrochloride, m.p. 188-192°;

4-(4-phenylsulfonylthiophene-3-sulfonyl)-1-[2-(4-fluorophenyl)ethyl]piperazine, hydrochloride, m.p. 158-159°;

4-(2-nitrophenylsulfonyl)-1-[2-(4-fluorophenyl)ethyl]piperazine, hydrochloride, m.p. 213-214°;

4-(5-bromo-6-chloropyridine-3-sulfonyl)-1-[2-(4-fluorophenyl)ethyl]piperazine, hydrochloride, m.p. 242-243°;

4-(2-aminophenylsulfonyl)-1-[2-(4-fluorophenyl)ethyl]piperazine, dihydrochloride/hydrate, m.p. 211-215°;

4-(6-chloroimidazo[2,1-b]thiazole-5-sulfonyl)-1-[2-(4-fluorophenyl)ethyl]piperazine, hydrochloride, m.p. 247-248°;

4-(1-acetyl-2,3-dihydroindole-5-sulfonyl)-1-[2-(4-fluorophenyl)ethyl]piperazine, m.p. 177-179°;

4-(2,3-dihydro-1H-indole-5-sulfonyl)-1-[2-(4-fluorophenyl)ethyl]piperazine, hydrochloride, m.p. 238-240°;

4-(indole-5-sulfonyl)-1-[2-(4-fluorophenyl)ethyl]piperazine, hydrochloride, m.p. 246-248°;

4-(1-methyl-1H-imidazole-4-sulfonyl)-1-[2-(4-fluorophenyl)ethyl]piperazine;

4-[1-(3-chloro-5-trifluoromethylpyridin-2-yl)pyrrole-3-sulfonyl)]-1-[2-(4-fluorophenyl)ethyl]piperazine, hydrochloride, m.p. 239-243°;

4-(isoquinoline-5-sulfonyl)-1-[2-(4-fluorophenyl)ethyl]piperazine, dihydrochloride, m.p. 243-244°.

### Example 1 a

4-(3-Cyano-1H-indole-5-sulfonyl)-1-[2-(4-fluorophenyl)ethyl]piperazine, m.p. 199-202°, is obtained in accordance with the following scheme.

The synthesis of the starting materials is described in J. Org. Chem. 53, 2047-2052 (1988).

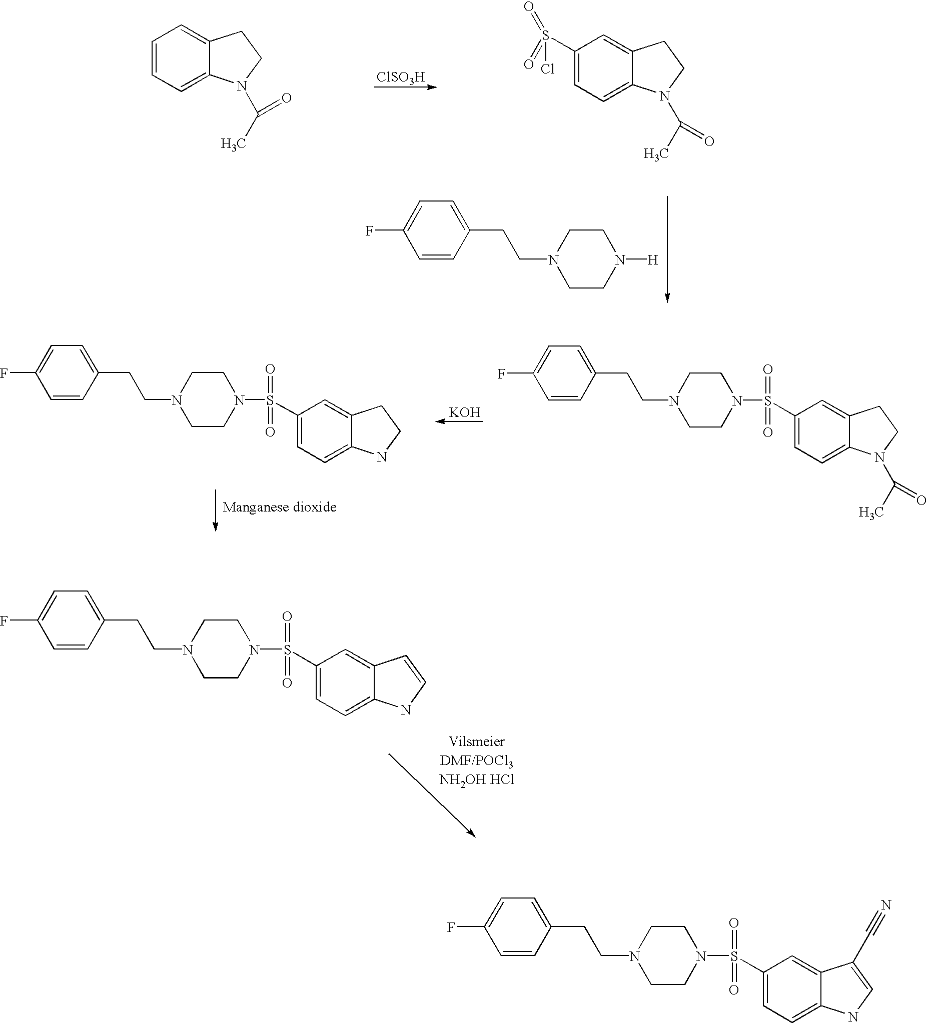

### Example 1 b

4-(3-Cyano-1H-indole-7-sulfonyl)-1-[2-(4-fluorophenyl)ethyl]piperazine is obtained in accordance with the following scheme.

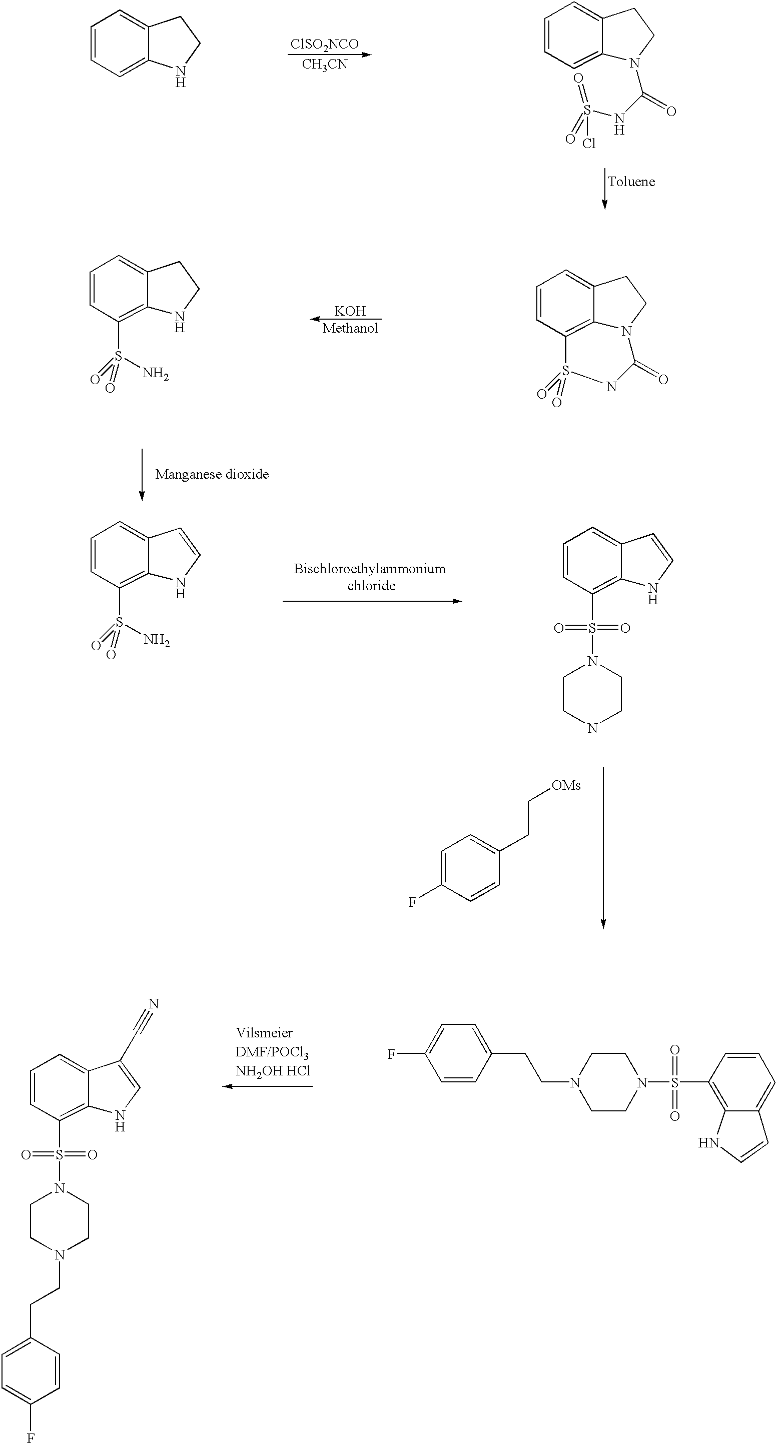

### Example 2

A solution of 500 mg of 2-chloro-6-(piperidin-4-ylsulfanyl)pyridine, hydrochloride, 500 mg of 2-(4-fluorophenyl)ethyl methanesulfonate and 500 mg of NaHCO₃ is stirred at 80° for 12 hours. After the mixture has been cooled, it is subjected to conventional work-up, giving 610 mg of 2-chloro-6-{1-[2-(4-fluorophenyl)ethyl]piperidin-4-ylsulfanyl}pyridine, hydrochloride (“AC”), m.p. 237-240°.

The following compounds are obtained analogously:

2-chloro-6-{1-[2-(2-fluorophenyl)ethyl]piperidin-4-ylsulfanyl}pyridine, hydrochloride, m.p. 182-184°;

2-chloro-6-{1-[2-(2-trifluoromethylphenyl)ethyl]piperidin-4-ylsulfanyl}pyridine, hydrochloride, m.p. 186-187°;

2-chloro-6-[1-(2-o-tolylethyl)piperidin-4-ylsulfanyl]pyridine, hydrochloride, m.p. 196-197°;

4-(4-fluorophenylsulfanyl)-1-[2-(4-fluorophenyl)ethyl]piperidine, hydrochloride, m.p. 195-196°;

4-(4-fluorophenylsulfanyl)-1-[2-(2-fluorophenyl)ethyl]piperidine, hydrochloride, m.p. 203-205°;

4-(4-fluorophenylsulfanyl)-1-[2-(2,4-difluorophenyl)ethyl]piperidine, hydrochloride, m.p. 204-206°;

4-phenylsulfanyl-1-[2-(4-fluorophenyl)ethyl]piperidine, hydrochloride, m.p. 209-211°;

naphthalen-2-ylsulfanyl-1-[2-(4-fluorophenyl)ethyl]piperidine, hydrochloride, m.p. 190-192°;

4-(4-methoxyphenylsulfanyl)-1-{2-(4-fluorophenyl)ethyl]piperidine, hydrochloride, m.p. 217-219°;

4-(3,4-dimethoxyphenylsulfanyl)-1-[2-(4-fluorophenyl)ethyl]piperidine, hydrochloride, m.p. 160-163°;

4-(2,4-dichlorophenylsulfanyl)-1-[2-(4-fluorophenyl)ethyl]piperidine, hydrochloride, m.p. 201-203°;

4-p-tolylsulfanyl-1-[2-(4-fluorophenyl)ethyl]piperidine, hydrochloride, m.p. 216-218°;

6-methoxy-2-{1-[2-(4-fluorophenyl)ethyl]piperidin-4-ylsulfanyl}pyridine, hydrochloride, m.p. 207-209°;

4-(4-trifluoromethoxyphenylsulfanyl)-1-[2-(4-fluorophenyl)ethyl]piperidine, hydrochloride, m.p. 188-189°;

4-(2,4-difluorophenylsulfanyl)-1-[2-(4-fluorophenyl)ethyl]piperidine, hydrochloride, m.p. 200-202°;

4-(4-trifluoromethylphenylsulfanyl)-1-[2-(4-fluorophenyl)ethyl]piperidine, hydrochloride, m.p. 193-195°;

4-(2-methoxyphenylsulfanyl)-1-[2-(4-fluorophenyl)ethyl]piperidine, hydrochloride, m.p. 223-226°;

4-(4-tert-butylphenylsulfanyl)-1-[2-(4-fluorophenyl)ethyl]piperidine, hydrochloride, m.p. 208-211°;

4-(2-fluorophenylsulfanyl)-1-[2-(4-fluorophenyl)ethyl]piperidine, hydrochloride, m.p. 209-210°;

4-(2-fluorophenylsulfanyl)-1-[2-(2,4-difluorophenyl)ethyl]piperidine, hydrochloride, m.p. 194-198°;

4-(2-fluorophenylsulfanyl)-1-[2-(3,4-difluorophenyl)ethyl]piperidine, 20 hydrochloride, m.p. 179-181°;

2-{1-[2-(4-fluorophenyl)ethyl]piperidin-4-ylsulfanyl}pyridine, hydrochloride, m.p. 243-245°;

4-(4-methylsulfanylphenylsulfanyl)-1-[2-(4-fluorophenyl)ethyl]piperidine, hydrochloride, m.p. 204-207°;

4-{1-[2-(4-fluorophenyl)ethyl]piperidin-4-ylsulfanyl}benzonitrile, hydrochloride, m.p. 206-207°;

4-(2,3-dichlorophenylsulfanyl)-1-[2-(4-fluorophenyl)ethyl]piperidine, hydrochloride, m.p. 197-199°;

8-{1-[2-(4-fluorophenyl)ethyl]piperidin-4-ylsulfanyl}quinoline, m.p. 88-90°;

4-{1-[2-(4-fluorophenyl)ethyl]piperidin-4-ylsulfanyl}pyridine, dihydrochloride;

2-{1-[2-(4-fluorophenyl)ethyl]piperidin-4-ylsulfanyl}benzothiazole, hydrochloride, m.p. 217-218°;

4-(2,4-dimethoxyphenylsulfanyl)-1-[2-(4-fluorophenyl)ethyl]piperidine, hydrochloride, m.p. 210-212°;

2-{1-[2-(4-fluorophenyl)ethyl]piperidin-4-ylsulfanyl}quinoline, hydrochloride, m.p. 257-259°;

4-{1-[2-(4-fluorophenyl)ethyl]piperidin-4-ylsulfanyl}7-trifluoromethylquinoline, dihydrochloride, m.p. 137-140°;

4-o-tolylsulfanyl-1-[2-(4-fluorophenyl)ethyl]piperidine, hydrochloride, m.p. 201-203°;

4-o-tolylsulfanyl-1-[2-(2-fluorophenyl)ethyl]piperidine, hydrochloride, m.p. 225-229°;

4-o-tolylsulfanyl-1-[2-(2,4-difluorophenyl)ethyl]piperidine, hydrochloride, m.p. 217-220°;

4-(2,4-dimethylphenylsulfanyl)-1-[2-(4-fluorophenyl)ethyl]piperidine, hydrochloride, m.p. 228-230°;

4-(2,4-dimethylphenylsulfanyl)-1-[2-(2-fluorophenyl)ethyl]piperidine, hydrochloride, m.p. 229-231°;

4-(2,4-dimethylphenylsulfanyl)-1-[2-(2,4-difluorophenyl)ethyl]piperidine, hydrochloride, m.p. 248-250°;

4-(thiazol-2-ylsulfanyl)-1-[2-(4-fluorophenyl)ethyl]piperidine, hydrochloride, m.p. 177-182°;

2-chloro-6-{1-[2-(5-chlorothiophen-2-yl)ethyl]piperidin-4-ylsulfanyl}pyridine, hydrochloride, m.p. 204-207°;

4-{1-[2-(5-chlorothiophen-2-yl)ethyl]piperidin-4-ylsulfanyl}benzonitrile, hydrochloride, m.p. 174-175°;

2-chloro-6{1-[2-(4-fluorophenyl)ethyl]piperidin-4-ylsulfanyl}pyridine, hydrochloride, m.p. 237-240°;

2-{1-[2-(4-fluorophenyl)ethyl]piperidin-4-ylsulfanyl}-1H-benzimidazole, hydrochloride, m.p. 234-235°;

4-(thiophen-2-ylsulfanyl)-1-[2-(4-fluorophenyl)ethyl]piperidine, dihydrochloride/hydrate, m.p. 213-214°;

4-{1-[2-(4-fluorophenyl)ethyl]piperidin-4-ylsulfanyl}phenol, hydrochloride, m.p. 196-199°;

2-{1-[2-(4-fluorophenyl)ethyl]piperidin-4-ylsulfanyl}phenol, hydrochloride, m.p. 190-192°;

3-{1-[2-(4-fluorophenyl)ethyl]piperidin-4-ylsulfanyl)-1H-indole, hydrochloride, m.p. 135°;

2-{1-[2-(4-fluorophenyl)ethyl]piperidin-4-ylsulfanyl}pyrimidine, hydrochloride, m.p. 205-209°;

4-(1-methyl-1H-imidazol-2-ylsulfanyl)-1-[2-(4-fluorophenyl)ethyl]piperidine, hydrochloride, m.p. 193-197°;

4-(4,5-dihydrothiazol-2-ylsulfanyl)-1-[2-(4-fluorophenyl)ethyl]piperidine, dihydrochloride/hydrate;

4-(2-chlorophenylsulfanyl)-1-[2-(4-fluorophenyl)ethyl]piperidine, hydrochloride, m.p. 205-207°;

4-(4-chlorophenylsulfanyl)-1-[2-(4-fluorophenyl)ethyl]piperidine, hydrochloride, m.p. 220-221°;

4-(2-methoxyphenylsulfanyl)-1-[2-(4-methoxyphenyl)ethyl]piperidine, hydrochloride, m.p. 205-207°;

4-(2-isopropylphenylsulfanyl)-1-[2-(4-fluorophenyl)ethyl]piperidine, hydrochloride, m.p. 203-205°;

2-{1-[2-(2,4-difluorophenyl)ethyl]piperidin-4-ylsulfanyl}phenol, hydrochloride, m.p. 92°;

2-{1-[2-(2-fluorophenyl)ethyl]piperidin-4-ylsulfanyl}phenol, m.p. 80°;

2-{1-[2-(3,4-difluorophenyl)ethyl]piperidin-4-ylsulfanyl}phenol, m.p. 100°;

4-(2-ethylphenylsulfanyl)-1-[2-(4-fluorophenyl)ethyl]piperidine, m.p. 200-203°;

4-(1-methyl-1H-tetrazol-5-ylsulfanyl)-1-[2-(4-fluorophenyl)ethyl]piperidine, hydrochloride, m.p. 193-195°;

4-(2,4,6-trimethylphenylsulfanyl)-1-[2-(4-fluorophenyl)ethyl]piperidine, hydrochloride, m.p. 248-250°;

4-(4-methyl-4H-[1,2,4]-triazol-3-ylsulfanyl)-1-[2-(4-fluorophenyl)ethyl]piperidine, hydrochloride, m.p. >260°;

8-{1-[2-(2,4-difluorophenyl)ethyl]piperidin-4-ylsulfanyl}quinoline, hydrochloride, m.p. 153-160°;

8-[1-(2-naphthalen-2-ylethyl)piperidin-4-ylsulfanyl]quinoline, dihydrochloride/hydrate, m.p. 217-219°;

8-[1-(2-naphthalen-1-ylethyl)piperidine-4-ylsulfanyl]quinoline, dihydrochloride/hydrate, m.p. 214-222°;

ethyl 2-{1-[2-(4-fluorophenyl)ethyl]piperidin-4-ylsulfanyl}benzoate, dihydrochloride/hydrate, m-p. 171-174°;

1-{1-[2-(4-fluorophenyl)ethyl]piperidin-4-ylsulfanyl}isoquinoline, hydrochloride, m.p. 282°;

2-{1-[2-(2,4-dichlorophenyl)ethyl]piperidin-4-ylsulfanyl}phenol, dihydrochloride, m.p. 254-259°;

2-[-(2-naphthalen-2-ylethyl)piperidin-4-ylsulfanyl]phenol, hydrochloride, m.p. 125°;

4-(4-acetylphenylsulfanyl)-1-[2-(4-fluorophenyl)ethyl]piperidine, hydrochloride, m.p. 208-210°;

8-{1-[2-(2-chloro-4-fluorophenyl)ethyl]piperidin-4-ylsulfanyl}quinoline, hydrochloride, m.p. 145-155°;

4-(2-methoxycarbonylmethylthiazol-4-ylsulfanyl)-1-[2-(4-fluorophenyl)ethyl]

4-(2-acetylphenylsulfanyl)-1-[2-(4-fluorophenyl)ethyl]piperidine, hydrochloride, m.p. 182-183°;

2-{1-[2-(4-fluorophenyl)ethyl]piperidin-4-ylsulfanyl}-6-methylpyridine, hydrochloride, m.p. 250-255°;

2-{1-[2-(2,4-difluorophenyl)ethyl]piperidin-4-ylsulfanyl}-1H-benzimidazole, dihydrochloride/dihydrate, m.p. 247-248°;

4-(2-methoxyphenylsulfanyl)-1-[2-(2,4-difluorofluorophenyl)ethyl]piperidine, dihydrochloride, m.p. 229-231°;

2-{1-[2-(2-chloro-4-fluorophenyl)ethyl]piperidin-4-ylsulfanyl}-1H-benzimidazole, hydrochloride;

2-{1-[2-(2-fluorophenyl)ethyl]piperidin-4-ylsulfanyl}-1H-benzimidazole, dihydrochloride, m.p. 190-194°;

4-{1-[2-(4-fluorophenyl)ethyl]piperidin-4-ylsulfanyl}-3-methyl-3H-imidazo[4,5-c]pyridine, dihydrochloride/hydrate, m.p. >250°;

4-(1H-indol-3-ylsulfanyl)-1-[2-(2,4-difluorophenyl)ethyl]piperidine, hydrochloride, m.p. 150°;

4-(1H-indol-3-ylsulfanyl)-1-[2-(2-fluorophenyl)ethyl]piperidine, hydrochloride, m.p. 190-193°;

4-(1H-indol-3-ylsulfanyl)-1-[2-(o-tolyl)ethyl]piperidine, hydrochloride, m.p. 200°;

4-{1[2-(4-fluorophenyl)ethyl]piperidin-4-ylsulfanyl}-1-methyl-1H-imidazo(4,5-c}pyridine, dihydrochloride, m.p. >280°;

4-{1-[2-(4-fluorophenyl)ethyl]piperidin-4-ylsulfanyl}-1H-imidazo[4,5-c]pyridine, trihydrochloride, m.p. >280°;

4-{1-[2-(4-fluorophenyl)ethyl]piperidin-4-ylsulfanyl}-2-methyl-1H-imidazo[4,5-c}pyridine, trihydrochloride, m.p. >145-152°;

2-{1-[2-(4-fluorophenyl)ethyl]piperidin-4-ylsulfanyl}-1H-imidazo[4,5-b]pyridine, dihydrochloride, m.p. 65-69°.

### Example 3

0.25 ml of hydrogen peroxide (30%) is added at room temperature to a solution of 390 mg of “AC” in 2.5 ml of glacial acetic acid, and the mixture is stirred for a further 12 hours. Conventional work-up gives 245 mg of 2-chloro-6-{1-[2-(4-fluorophenyl)ethyl]piperidine-4-sulfinyl}pyridine, hydrochloride, m.p. 208°, and 60 mg of 2-chloro-6-{1-[2-(4-fluorophenyl) ethyl]piperidine-4-sulfonyl}pyridine, hydrochloride, m.p. 208°.

The following compounds are obtained analogously:

4-(4-fluorophenylsulfinyl)-1-[2-(4-fluorophenyl)ethyl]piperidine, hydrochloride, m.p. 225°;

4-(4-fluorophenylsulfonyl)-1-[2-(4-fluorophenyl)ethyl]piperidine, hydrochloride, m.p. 243°;

4-(4-fluorophenylsulfonyl)-1-[2-(2-fluorophenyl)ethyl]piperidine, hydrochloride, m.p. 240°;

4-(4-fluorophenylsulfonyl)-1-[2-(2,4-difluorophenyl)ethyl]piperidine, hydrochloride, m.p. 253°;

4-(phenylsulfonyl)-1-[2-(4-fluorophenyl)ethyl]piperidine, hydrochloride, m.p. 246-247°;

4-(phenylsulfinyl)-1-[2-(4-fluorophenyl)ethyl]piperidine, hydrochloride, m.p. 222-224°;

4-(2-naphthylsulfinyl)-1-[2-(4-fluorophenyl)ethyl]piperidine, hydrochloride, m.p. 197-199°;

4-(2-naphthylsulfonyl)-1-[2-(4-fluorophenyl)ethyl]piperidine, hydrochloride, m.p. 253-255°;

4-(4-methoxyphenylsulfinyl)-1-[2-(4-fluorophenyl)ethyl]piperidine, hydrochloride, m.p. 228-230°;

4-(4-methoxyphenylsulfonyl)-1-[2-(4-fluorophenyl)ethyl]piperidine, hydrochloride, m.p. 232-234°;

4-(3,4-dimethoxyphenylsulfinyl)-1-[2-(4-fluorophenyl)ethyl]piperidine, hydrochloride, m.p. 180-182°;

4-(3,4-dimethoxyphenylsulfonyl)-1-[2-(4-fluorophenyl)ethyl]piperidine, hydrochloride, m.p. 194-195°;

4-(2,4-dichlorophenylsulfinyl)-1-[2-(4-fluorophenyl)ethyl]piperidine, hydrochloride, m.p. 220-223°;

4-(2,4-dichlorophenylsulfonyl)-1-[2-(4-fluorophenyl)ethyl]piperidine, hydrochloride, m.p. 271-275°;

4-(4-tolylsulfinyl)-1-[2-(4-fluorophenyl)ethyl]piperidine, hydrochloride, m.p. 223-224°;

4-(4-tolylsulfonyl)-1-[2-(4-fluorophenyl)ethyl]piperidine, hydrochloride, m.p. 265-267°;

4-(2,4-difluorophenylsulfinyl)-1-[2-(4-fluorophenyl)ethyl]piperidine, hydrochloride, m.p. 222-223°;

4-(2,4-difluorophenylsulfonyl)-1-[2-(4-fluorophenyl)ethyl]piperidine, hydrochloride, m.p. 240-241°;

4-(2-fluorophenylsulfonyl)-1-[2-(4-fluorophenyl)ethyl]piperidine, hydrochloride, m.p. 228-230°;

4-(2-fluorophenylsulfonyl)-1-[2-(2,4-difluorophenyl)ethyl]piperidine, hydrochloride, m.p. 249-251°;

4-(2-fluorophenylsulfonyl)-1-[2-(3,4-difluorophenyl)ethyl]piperidine, hydrochloride, m.p. 203-205°;

6-methoxy-2-{1-[2-(4-fluorophenyl)ethyl]piperidin-4-ylsulfonyl}pyridine, hydrochloride, m.p. 202-203°;

6-methoxy-2-{1-[2-(4-fluorophenyl)ethyl]piperidin-4-ylsulfinyl}pyridine, hydrochloride, m.p. 186-188°;

4-(2-fluorophenylsulfinyl)-1-[2-(4-fluorophenyl)ethyl]piperidine, hydrochloride, m.p. 200-202°;

4-(2-fluorophenylsulfinyl)-1-[2-(2,4-difluorophenyl)ethyl]piperidine, hydrochloride, m.p. 183-185°;

4-(2-fluorophenylsulfinyl)-1-[2-(3,4-difluorophenyl)ethyl]piperidine, hydrochloride, m.p. 191-193°;

4-(4-trifluoromethylphenylsulfonyl)-1-[2-(4-fluorophenyl)ethyl]piperidine, hydrochloride, m.p. 270-272°;

4-(4-trifluoromethylphenylsulfinyl)-1-[2-(4-fluorophenyl)ethyl]piperidine, hydrochloride, m.p. 218-219°;

4-(4-trifluoromethoxyphenylsulfinyl)-1-[2-(4-fluorophenyl)ethyl]piperidine, hydrochloride, m.p. 210-211°;

4-(4-trifluoromethoxyphenylsulfonyl)-1-[2-(4-fluorophenyl)ethyl]piperidine, hydrochloride, m.p. 249-251°;

4-(2-methoxyphenylsulfonyl)-1-[2-(4-fluorophenyl)ethyl]piperidine, hydrochloride, m.p. 245-247°;

4-(2-methoxyphenylsulfinyl)-1-[2-(4-fluorophenyl)ethyl]piperidine, hydrochloride, m.p. 208-210°;

4-(4-tert-butylphenylsulfonyl)-1-[2-(4-fluorophenyl)ethyl]piperidine, hydrochloride, m.p. 274-276°;

4-(4-tert-butylphenylsulfinyl)-1-[2-(4-fluorophenyl)ethyl]piperidine, hydrochloride, m.p. 226-228°;

4-{1-[2-(4-fluorophenyl)ethyl]piperidin-4-ylsulfonyl}benzonitrile, hydrochloride, m.p. >260°;

2-{1-2-(4-fluorophenyl)ethyl]piperidin-4-ylsulfonyl}pyridine, hydrochloride, m.p. 228-230°;

2-{1-[2-(4-fluorophenyl)ethyl]piperidin-4-ylsulfinyl}pyridine, hydrochloride, m.p. 205-210°;

4-(2,3-dichlorophenylsulfonyl)-1-[2-(4-fluorophenyl)ethyl]piperidine,

4-(2,3-dichlorophenylsulfinyl)-1-[2-(4-fluorophenyl)ethyl]piperidine, hydrochloride, m.p. 227-228°;

4-(2-fluorophenylmethanesulfonyl)-1-[2-(4-fluorophenyl)ethyl]piperidine, hydrochloride, m.p. 268-270°;

4-(2-fluorophenylmethanesulfinyl)-1-[2-(4-fluorophenyl)ethyl]piperidine, hydrochloride, m.p. 198-199°;

4-{1-[2-(4-fluorophenyl)ethyl]piperidin-4-ylsulfonyl}pyridine, dihydrochloride, m.p. 228-240°;

4-{1-[2-(4-fluorophenyl)ethyl]piperidin-4-ylsulfinyl}pyridine, dihydrochloride, m.p. 166-170°;

8-{1-[2-(4-fluorophenyl)ethyl]piperidin-4-ylsulfonyl}quinoline, hydrochloride, m.p. 255-265°;

8-{1-[2-(4-fluorophenyl)ethyl]piperidin-4-ylsulfinyl}quinoline, hydrochloride, m.p. 210°;

6{1-[2-(4-fluorophenyl)ethyl]piperidin-4-ylsulfonyl}nicotinamide, hydrochloride;

4-(4-methanesulfinylphenylsulfonyl)-1-[2-(4-fluorophenyl)ethyl]piperidine, hydrochloride, m.p. 180-185°;

2-{1-[2-(4-fluorophenyl)ethyl]piperidin-4-ylsulfonyl}quinoline, hydrochloride, m.p. 238-240°;

2-{1-[2-(4-fluorophenyl)ethyl]piperidin-4-ylsulfinyl}quinoline, hydrochloride, m.p. 210-213°;

4-(2,4-dimethoxyphenylsulfinyl)-1-[2-(4-fluorophenyl)ethyl]piperidine, hydrochloride, m.p. 208-210°;

4-(2,4-dimethoxyphenylsulfonyl)-1-[2-(4-fluorophenyl)ethyl]piperidine, hydrochloride, m.p. 238°;

2-{1-[2-(4-fluorophenyl)ethyl]piperidin-4-ylsulfonyl}benzothiazole, hydrochloride, m.p. 233-234°;

4-(4-methanesulfinylphenylsulfinyl)-1-[2-(4-fluorophenyl)ethyl]piperidine, hydrochloride, m.p. 180-185°;

4-[2-(4-phenylsulfonylpiperidin-1-yl)ethyl]pyridine, dihydrochloride, m.p. 187-193°;

4-[2-(4-phenylsulfinylpiperidin-1-yl)ethyl]pyridine, dihydrochloride, m.p. 153-155°;

4-{2-[4-(3,4-dimethoxyphenylsulfonyl)piperidin-1-yl]ethyl}pyridine, dihydrochloride, m.p. 125-135°;

4-{2-[4-(3,4-dimethoxyphenylsulfinyl)piperidin-1-yl]ethyl}pyridine, dihydrochloride, m.p. 149-155°;

4-{2-[4-(tolyl-4-sulfinyl)piperidin-1-yl]ethyl}pyridine, dihydrochloride, m.p. 210-214°;

4-{2-[4-(2-methoxyphenylsulfonyl)piperidin-1-yl]ethyl}pyridine, dihydrochloride, m.p. 200-218°;

4-{2-[4-(2-methoxyphenylsulfinyl)piperidin-1-yl]ethyl}pyridine, dihydrochloride, m.p. 214-222°;

4-{1-[2-(5-chlorothiophen-2-yl)ethyl]piperidin-4-ylsulfonyl}benzonitrile, hydrochloride, m.p. >250°;

4-{1-[2-(5-chlorothiophen-2-yl)ethyl]piperidin-4-ylsulfinyl}benzonitrile, hydrochloride, m.p. 200-202°;

1-[2-(4-fluorophenyl)ethyl]-4-(2-methylphenylsulfonyl)piperidine, hydrochloride, m.p. 221-223°;

1-[2-(4-fluorophenyl)ethyl]4-(2-methylphenylsulfinyl)piperidine, hydrochloride, m.p. 214-216°;

1-[2-(2-fluorophenyl)ethyl]-4-(2-methylphenylsulfonyl)piperidine, hydrochloride, m.p. 218-221°;

1-[2-(2-fluorophenyl)ethyl]-4-(2-methylphenylsulfinyl)piperidine, hydrochloride, m.p. 202-204°;

1-[2-(2,4-difluorophenyl)ethyl]-4-(2-methylphenylsulfonyl)piperidine, hydrochloride, m.p. 235-240°;

1-[2-(2,4-difluorophenyl)ethyl]-4-(2-methylphenylsulfinyl)piperidine, hydrochloride, m.p. 216-217°;

1-[2-(4-fluorophenyl)ethyl]-4-(2,4-dimethylphenylsulfonyl)piperidine, hydrochloride, m.p. 247-248°;

1-[2-(4-fluorophenyl)ethyl]-4-(2,4-dimethylphenylsulfinyl)piperidine, hydrochloride, m.p. 237-238°;

1-[2-(2-fluorophenyl)ethyl]-4-(2,4-dimethylphenylsulfonyl)piperidine, hydrochloride, m.p. 242-244°;

1-[2-(2-fluorophenyl)ethyl]-4-(2,4-dimethylphenylsulfinyl)piperidine, hydrochloride, m.p. 230-232°;

2-{1-[2-(4-fluorophenyl)ethyl]piperidine-4-sulfonyl}phenol, hydrochloride, m.p. 275°;

2-{1-[2-(4-fluorophenyl)ethyl]piperidine-4-sulfinyl}phenol, hydrochloride, m.p. 262°;

4-{1-[2-(4-fluorophenyl)ethyl]piperidine-4-sulfonyl}phenol, hydrochloride, m.p. 145°;

4-{1-[2-(4-fluorophenyl)ethyl]piperidine-4-sulfinyl}phenol, hydrochloride, m.p. 130-135°;

1-[2-(2,4-difluorophenyl)ethyl]-4-(2,4-dimethylphenylsulfonyl)piperidine, hydrochloride, m.p. 255-257°;

1-[2-(2,4-difluorophenyl)ethyl]-4-(2,4-dimethylphenylsulfinyl)piperidine, hydrochloride, m.p. 236-238°;

1-[2-(4-methoxyphenyl)ethyl]-4-(2-methoxyphenylsulfonyl)piperidine, hydrochloride, m.p. 258-260°;

1-[2-(4-fluorophenyl)ethyl]-4-(2,4,6-trimethylphenylsulfonyl)piperidine, hydrochloride, m.p. 280-283°;

1-[2-(4-fluorophenyl)ethyl]-4-(2,4,6-trimethylphenylsulfinyl)piperidine, hydrochloride, m.p. 220-223°;

2-{1-[2-(2,4-difluorophenyl)ethyl]piperidine-4-sulfonyl}phenol, hydrochloride, m.p. >250°;

1-[2-(2,4-difluorophenyl)ethyl]-4-(2-methoxyphenylsulfonyl)piperidine, hydrochloride, m.p. 229-232°;

1-[2-(2,4-difluorophenyl)ethyl]-4-(2-methoxyphenylsulfinyl)piperidine, hydrochloride, m.p. 210-214°;

1-[2-(4-fluorophenyl)ethyl]-4-(thiazole-2-sulfonyl)piperidine, hydrochloride, m.p. 197-198°;

1-[2-(4-fluorophenyl)ethyl]-4-(thiazole-2-sulfinyl)piperidine, hydrochloride, m.p. 221-223°;

1-[2-(4-fluorophenyl)ethyl]-4-(thiophene-2-sulfonyl)piperidine, hydrochloride, m.p. 239-241°;

1-[2-(4-fluorophenyl)ethyl]-4-(thiophene-2-sulfinyl)piperidine, hydrochloride, m.p. 206-207°;

1-[2-(4-fluorophenyl)ethyl]-4-(pyrimidine-2-sulfonyl)piperidine, hydrochloride, m.p. 233-234°;

1-[2-(4-fluorophenyl)ethyl]-4-(pyrimidine-2-sulfinyl)piperidine, hydrochloride, m.p. 173-176°;

1-[2-(4-fluorophenyl)ethyl]-4-(1-methyl-1H-imidazole-2-sulfonyl)piperidine, dihydrochloride/hydrate, m.p. 237-239°;

1-[2-(4-fluorophenyl)ethyl]-4-(1-methyl-1H-imidazole-2-sulfinyl)piperidine, dihydrochloride/hydrate, m.p. 199-202°;

1-[2-(4-fluorophenyl)ethyl]-4-(2-chlorophenylsulfonyl)piperidine, hydrochloride, m.p. 252-253°;

1-[2-(4-fluorophenyl)ethyl]-4-(2-chlorophenylsulfinyl)piperidine, hydrochloride, m.p. 209-210°;

1-[2-(4-fluorophenyl)ethyl]-4-(4-chlorophenylsulfonyl)piperidine, hydrochloride, m.p. 242-246°;

1-[2-(4-fluorophenyl)ethyl]-4-(2-isopropylphenylsulfonyl)piperidine, hydrochloride/hydrate, m.p. 231-233°;

1-[2-(4-fluorophenyl)ethyl]-4-(2-isopropylphenylsulfinyl)piperidine, hydrochloride, m.p. 200-203°;

1-[2-(4-fluorophenyl)ethyl]-4-(2-ethylphenylsulfonyl)piperidine, hydrochloride, m.p. 229-231°;

1-[2-(4-fluorophenyl)ethyl]-4-(2-ethylphenylsulfinyl)piperidine, hydrochloride/hydrate, m.p. 204-206°;

1-[2-(4-fluorophenyl)ethyl]-4-(1-methyl-1H-tetrazole-5-sulfinyl)piperidine, hydrochloride, m.p. 161-163°;

4-(2-acetylphenylsulfonyl)-1-[2-(4-fluorophenyl)ethyl]piperidine, hydrochloride, m.p. 224-226°;

4-(4-acetylphenylsulfonyl)-1-[2-(4-fluorophenyl)ethyl]piperidine, hydrochloride, m.p. 242-244°;

4-(4-acetylphenylsulfinyl)-1-[2-(4-fluorophenyl)ethyl]piperidine, hydrochloride, m.p. 225-227°;

4-(2-ethoxycarbonylphenylsulfonyl)-1-[2-(4-fluorophenyl)ethyl]piperidine, hydrochloride, m.p. 204-209°;

4-(6-chloropyridine-2-sulfinyl)-1-[2-(4-fluorophenyl)ethyl]piperidine, hydrochloride, m.p. 208°;

4-(6-chloropyridine-2-sulfonyl)-1-[2-(4-fluorophenyl)ethyl]piperidine, hydrochloride, m.p. 208°;

4-(1H-indole-3-sulfonyl)-1-[2-(2,4-difluorophenyl)ethyl]piperidine, hydrochloride, (m.p.. 150-200°);

4-(1H-indole-3-sulfonyl)-1-[2-(4-fluorophenyl)ethyl]piperidine, hydrochloride, (m.p. 150-200°);

4-(3-methyl-3H-imidazo[4,5-c]pyridine-4-sulfonyl)-1-[2-(4-fluorophenyl)ethyl]piperidine, dihydrochloride, m.p. 227-229°;

4-(3-methyl-3H-imidazo[4,5-c]pyridine-4-sulfinyl)-1-[2-(4-fluorophenyl)ethyl]piperidine, dihydrochloride, m.p. 173-175°;

4-(1H-benzimidazole-2-sulfonyl)-1-[2-(4-fluorophenyl)ethyl]piperidine, dihydrochloride, m.p. 110-125°;

4-(1-methyl-1H-imidazo[4,5-c]pyridine-4-sulfonyl)-1-[2-(4-fluorophenyl)-ethyl]piperidine, dihydrochloride/hydrate, m.p. 186-192°;

4-(1-methyl-1H-imidazo[4,5-c]pyridine-4-sulfinyl)-1-[2-(4-fluorophenyl)-ethyl]piperidine, dihydrochloride/dihydrate, m.p. 130-135°;

4-(2-methyl-1H-imidazo[4,5-c]pyridine-4-sulfinyl)-1-[2-(4-fluorophenyl)-ethyl]piperidine, dihydrochloride/dihydrate, m.p. 210-220°;

4-(Isoquinoline-1-sulfonyl)-1-[2-(4-fluorophenyl)ethyl]piperidine, hydrochloride, m.p. 237-240°;

4-(quinoline-8-sulfonyl)-1-[2-(2,4-dichlorophenyl)ethyl]piperidine, dihydrochloride/hydrate, m.p. 210-215°;

4-(quinoline-8-sulfinyl)-1-[2-(2,4-dichlorophenyl)ethyl]piperidine, dihydrochloride, m.p. 215-217°;

4-(quinoline-8-sulfonyl)-1-[2-(naphthalen-2-yl)ethyl]piperidine, hydrochloride, m.p. >280°;

4-(quinoline-8-sulfinyl)-1-[2-(naphthalen-2-yl)ethyl]piperidine, hydrochloride, m.p. 205-213°;

4-(quinoline-8-sulfonyl)-1-[2-(2-chloro-4-fluorophenyl)ethyl]piperidine, dihydrochloride/hydrate, m.p. 150-164°;

4-(1H-benzimidazole-2-sulfonyl)-1-[2-(2-chloro-4-fluorophenyl)ethyl]piperidine, dihydrochloride.

The examples below relate to pharmaceutical preparations:

### Example A

Injection Vials

A solution of 100 g of an active ingredient of the formula I and 5 g of disodium hydrogenphosphate in 3 I of bidistilled water is adjusted to pH 6.5 using 2N hydrochloric acid, sterile filtered, transferred into injection vials, lyophilized under sterile conditions and sealed under sterile conditions. Each injection vial contains 5 mg of active ingredient.

### Example B

Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### Example C

Solution

A solution is prepared from 1 g of an active ingredient of the formula I, 9.38 g of NaH₂PO₄×2 H₂O, 28.48 g of Na₂HPO₄×12 H₂O and 0.1 g of benzalkonium chloride in 940 ml of bidistilled water. The pH is adjusted to 6.8, and the solution is made up to 1 I and sterilized by irradiation. This solution can be used in the form of eye drops.

### Example D

Ointment

500 mg of an active ingredient of the formula I are mixed with 99.5 g of Vaseline under aseptic conditions.

### Example E

Tablets

A mixture of 1 kg of an active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

Coated tablets

Tablets are pressed analogously to Example E and subsequently coated in a conventional manner with a coating of sucrose, potato starch, talc, tragacanth and dye.

### Example G

Capsules

2 kg of an active ingredient of the formula I are introduced into hard gelatine capsules in a conventional manner in such a way that each capsule contains 20 mg of the active ingredient.

### Example H

Ampoules

A solution of 1 kg of an active ingredient of the formula I in 60 1 I of bidistilled water is transferred into ampoules, lyophilized under aseptic conditions and sealed under sterile conditions. Each ampoule contains 10 mg of active

1. Compounds of the formula I 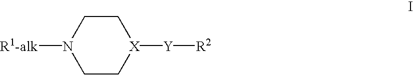
in which
R¹ and R² are each, independently of one another, a phenyl or naphthyl radical which is unsubstituted or substituted by R³, R⁴ and/or R⁵ or are Het¹,
R³, R⁴ and R⁵ are each, independently of one another, Hal, A, OA, OH, CN, NO₂, NH₂, NHA, NA₂, NH-acyl, acyl, —SA, —SOA, SO₂A, COOA or phenyl,
X is CH or N,
Y is SO₂ if X=N or S, SO or SO₂ if X=CH,
Het¹ is an unsaturated heterocyclic ring system which is unsubstituted or monosubstituted, disubstituted or trisubstituted by Hal, A, CN, CONH₂, CH₂COOA, phenyl-SO₂, acyl, OA or OH and which contains one, two or three identical or different hetero atoms, such as nitrogen, oxygen and sulfur,
A is alkyl having 1-6 carbon atoms,
alk is alkylene having 1-6 carbon atoms, and
Hal is F, Cl, Br or I,
where Het¹≠2,1,3-benzoxadiazolyl or 2,1,3-benzothiadiazolyl, and their physiologically acceptable salts and solvates.

2. Compounds according to claim 1
a) 8-{4-[2-(4-fluorophenyl)ethyl]piperazine-1-sulfonyl)quinoline;
b) 4-(4-fluorophenylsulfonyl)-1-[2-(4-fluorophenyl)ethyl]piperidine;
c) 2-chloro-6-{1-[2-(4-fluorophenyl)ethyl]piperidin-4-sulfonyl}pyridine;
d) 4-(2-methoxyphenylsulfanyl)-1-[2-(4-fluorophenyl)ethyl]-piperidine
e) 4-(4-methylphenylsulfinyl)-1-[2-(4-fluorophenyl)ethyl]piperidine;
f) 4-(3-cyano-1H-indole-5-sulfonyl)-1-[2-(4-fluorophenyl)ethyl]-piperazine
and their physiologically acceptable salts and solvates.

3. Process for the preparation of compounds of the formula I according to claim 1 in which X is N, characterized in that
a) a compound of the formula II 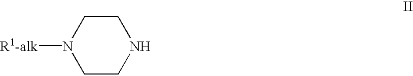
in which R¹ and alk are as defined in claim 1, is reacted with a compound of the formula III
R²—Y—L III
in which L is Cl, Br, I or a free or reactively functionally modified OH group,
and R² and Y are as defined in claim 1,
or
b) if desired one of the radicals R¹ and/or R² is converted into another radical R¹ and/or R² by, for example, cleaving an OA group to form an OH group and/or converting a CHO group into a CN group,
and/or
a resultant base of the formula I is converted into one of its salts by treatment with an acid.

4. Process for the preparation of compounds of the formula I according to claim 1 in which X is CH, characterized in that
a) a compound of the formula IV 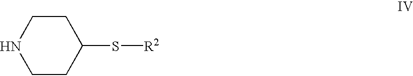
in which R² is as defined in claim 1, is reacted with a compound of the formula V
R¹-alk-L V
in which L is Cl, Br, I or a free or reactively functionally modified OH group,
and R¹ and alk are as defined in claim 1,
and the product is subsequently oxidized,
or
b) if desired one of the radicals R¹ and/or R² is converted into another radical R¹ and/or R² by, for example, cleaving an OA group to form an OH group and/or converting a CHO group into a CN group,
and/or
a resultant base of the formula I is converted into one of its salts by treatment with an acid.

5. Compounds of the formula I according to claim 1, and their physiologically acceptable salts and solvates, as medicaments.

6. Compounds of the formula I 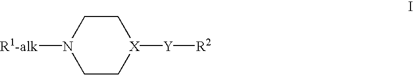
in which
R¹ and R² are each, independently of one another, a phenyl or naphthyl radical which is unsubstituted or substituted by R³, R⁴ and/or R⁵ or are Het¹,
R³, R⁴ and R⁵ are each, independently of one another, Hal, A, OA, OH, CN, NO₂, NH₂, NHA, NA₂, NH-acyl, acyl, —SA, —SOA, SO₂A, COOA or phenyl,
X is CH or N,
Y is SO₂ if X=N or
S, SO or SO₂ if X=CH,
Het¹ is an unsaturated heterocyclic ring system which is unsubstituted or monosubstituted, disubstituted or trisubstituted by Hal, A, CN, CONH₂, CH₂COOA, phenyl-SO₂, acyl, OA or OH and which contains one, two or three identical or different hetero atoms, such as nitrogen, oxygen and sulfur,
A is alkyl having 1-6 carbon atoms,
alk is alkylene having 1-6 carbon atoms, and
Hal is F, Cl, Br or I,
and their physiologically acceptable salts and solvates as medicaments having a 5-HT_{2A} receptor-antagonistic action.

7. Medicament according to claim 5 or 6 for the treatment of psychoses, schizophrenia, depression, neurological disorders, memory disorders, Parkinson's disease, amyotrophic lateral sclerosis, Alzheimer's disease, Huntington's disease, eating disorders, such as bulimia, anorexia nervosa, premenstrual syndrome and/or for positively influencing obsessive-compulsive disorder (OCD).

8. Pharmaceutical preparation comprising at least one medicament according to claim 5 or 6 and optionally vehicles and/or auxiliaries and optionally other active ingredients.

9. Use of compounds according to claim 1 and/or their physiologically acceptable salts and solvates for the preparation of a medicament having a 5-HT_{2A} receptor-antagonistic action.

10. Use according to claim 9 for the preparation of a medicament for the treatment of psychoses, schizophrenia, depression, neurological disorders, memory disorders, Parkinson's disease, amyotrophic lateral sclerosis, Alzheimer's disease, Huntington's disease, eating disorders, such as bulimia, anorexia nervosa, premenstrual syndrome and/or for positively influencing obsessive-compulsive disorder (OCD).
